(19) United States
(12) Patent Application Publication
(10) Pub. No.: US 2026/0366670 A1
(43) Pub. Date: Nov. 19, 2026

(54) OPERATION PANEL ASSEMBLY AND MEDICAL DEVICE

(71) Applicant: GE Precision Healthcare LLC, Waukesha, WI (US)

(72) Inventors: Lu Jin, Wuxi (CN); Jonathan Boomgaarden, Wauwatosa, WI (US); Michael Macdonald, Wauwatosa, WI (US); Qiang Yao, Wuxi (CN); Todd Schueneman, Wauwatosa, WI (US); Ross Stalter, Wauwatosa, WI (US)

(21) Appl. No.: 19/393,201

(22) Filed: Nov. 18, 2025

(30) Foreign Application Priority Data
Nov. 20, 2024 (CN) 202411666443.4

(51) Int. Cl. F16M 11/04; A61B 8/00; A61B 50/13; F16M 11/08; F16M 11/18; F16M 11/20; F16M 11/28; F16M 11/42

(52) U.S. Cl. CPC F16M 11/048; A61B 8/4405; A61B 50/13; F16M 11/08; F16M 11/18; F16M 11/2014; F16M 11/28; F16M 11/42; F16M 2200/021; F16M 2200/025

(57) ABSTRACT

An operation panel assembly, including: a support assembly; an arm assembly comprising a first arm, a second arm, and a third arm, the first arm being connected to the support assembly, and the second arm and the third arm being telescopically connected with respect to the first arm; and an operation panel connected to the third arm.

### CROSS-REFERENCE TO RELATED APPLICATION

This application claim priority to Chinese Patent Application No. 202411666443.4, which was file on Nov. 20, 2024 at the Chinese Patent Office. The entire contents of the above-listed application are incorporated by reference herein in their entirety.

### TECHNICAL FIELD

The present application relates to the field of mechanical apparatuses, and relates in particular to an operation panel assembly and a medical device including the operation panel assembly.

### BACKGROUND

An operation panel is a widely used apparatus. Depending on different usage scenarios, the operation panel can be given different functions. For example, it may be integrated with a user input apparatus (e.g., a keyboard, a touch screen, etc.) to provide a user with human-machine interactive functionality. Alternatively, the operation panel may be used for placement of a system for the user to operate. The operation panel is also often used in the medical field. For example, carts for carrying different types of medical apparatuses or certain medical apparatuses themselves (such as an ultrasound machine) include an operation panel.

The optimal distance or angle of the operation panel relative to the user may be different for different users or the same user in different usage scenarios. Thus, the operation panel may be configured to be adjustable to adapt to different needs. For example, the operation panel is often disposed to telescopable and/or rotatable in a horizontal plane, thereby adapting to different needs of different distances and/or angles. However, considering the reliability of the device, the stroke of telescoping and/or rotation may need to be limited to a greater extent. This can ensure that the load-bearing capacity of the operation panel is within an acceptable range even when extended to the farthest distance. However, this may compromise user comfort for the operation panel.

### SUMMARY OF THE INVENTION

In view of at least the foregoing, some embodiments of the present application provide an operation panel assembly, comprising: a support assembly; an arm assembly comprising a first arm, a second arm, and a third arm; the first arm being connected to the support assembly; the second arm and the third arm being telescopically connected with respect to the first arm; and an operation panel connected to the third arm.

Optionally, the arm assembly further comprises a synchronization apparatus through which the first arm, the second arm, and the third arm join one another to achieve synchronization of a telescoping process of the second arm and the third arm with respect to the first arm.

Optionally, the synchronization apparatus comprises: a first rack disposed on the first arm; a second rack disposed on the third arm, the first rack and the second rack having a gap therebetween; and a gear disposed on the second arm and engaged in the gap; wherein the first arm, the second arm, and the third arm join one another through the first rack, the gear, and the second rack.

Optionally, the synchronization apparatus comprises: a transmission belt tensioned by a first transmission pulley and a second transmission pulley disposed on the second arm; a first fixing apparatus separately fixed to the first arm and one side of the transmission belt; and a second fixing apparatus separately fixed to the third arm and the other side of the transmission belt.

Optionally, the support assembly comprises a base and a lifting apparatus. The lifting apparatus is connected to the first arm to drive the operation panel to lift by means of the first arm, the second arm, and the third arm.

Optionally, the operation panel and the third arm are rotatably connected; and/or, the support assembly and the first arm are rotatably connected.

Optionally, the operation panel assembly further comprises an attachment apparatus connected to the support assembly and extending upward to a height not lower than the height of the operation panel; wherein extension strokes of the second arm and the third arm are configured such that rotational steric hindrance of the attachment apparatus to the operation panel can be eliminated.

Optionally, the attachment apparatus comprises a rear handle.

Optionally, the first arm comprises a first flat plate portion and a first pair of oppositely disposed side walls; the second arm comprises a second flat plate portion and a second pair of oppositely disposed side walls; the third arm comprises a third flat plate portion and a third pair of oppositely disposed side walls; and the first pair of side walls, the second pair of side walls, and the third pair of side walls are disposed in a nested manner to guide telescoping of the second arm and the third arm.

Optionally, a first pair of guide rails and a first pair of guide wheels are disposed between the first pair of side walls and the second pair of side walls, respectively, the first pair of guide wheels being received in the first pair of guide rails; and a second pair of guide rails and a second pair of guide wheels are disposed between the second pair of side arms and the third pair of side arms, respectively, the second pair of guide wheels being received in the second pair of guide rails.

Optionally, the second pair of side walls further comprises a third pair of guide wheels disposed at bottoms of the second pair of side walls and between the first pair of side walls and the third pair of side walls.

Optionally, the operation panel assembly further comprises a buffer assembly comprising a first buffer member and a second buffer member that are oppositely disposed, the first buffer member and the second buffer member being disposed on any one arm and any other arm among the first arm, the second arm, and the third arm, respectively.

Optionally, the first buffer member comprises two first buffer members disposed at front and rear ends of the one arm, and the second buffer member is disposed on the other arm and located between the two first buffer members.

Optionally, the operation panel and the third arm are rotatably connected. Also, the operation panel assembly further comprises: a first locking member disposed between the operation panel and the third arm, the first locking member being operable to lock the operation panel and the third arm, so as to achieve rotational locking between the operation panel and the third arm.

Optionally, the operation panel assembly further comprises a second locking member disposed between the support assembly and the second arm, the second locking member being operable to lock the support assembly and the second arm, so as to achieve telescopic locking of the second arm.

Optionally, the support assembly and the first arm are rotatably connected, whereby the second locking member achieves rotational locking between the first arm and the support assembly while telescopically locking the second arm.

Further embodiments of the present application further provide a medical device, including any one of the operation panel assemblies described above.

Optionally, the medical device comprises at least one of a medical cart and an ultrasound imaging apparatus.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, where:

FIG. 1 is a schematic view of an apparatus including an operation panel in the prior art;

FIG. 2 is an apparatus including an operation panel assembly in some embodiments of the present application;

FIG. 3 is a perspective view of an operation panel assembly in further embodiments of the present application;

FIG. 4 is a schematic view of the connection among the three arms of the operation panel assembly of FIG. 3;

FIG. 5 shows a bottom view of an arm assembly in some embodiments of the present application;

FIG. 6 shows a bottom view of an arm assembly in some other embodiments of the present application;

FIG. 7 shows a schematic view of an operation panel assembly in a first state in some embodiments of the present application;

FIG. 8 shows a schematic view of the operation panel assembly in a second state in some embodiments of the present application;

FIG. 9 shows a partial schematic view of an operation panel assembly including a first locking member in some embodiments of the present application;

FIG. 10 shows a partial schematic view of an operation panel assembly including a second locking member in some embodiments of the present application; and

FIG. 11 shows a schematic view of an ultrasound imaging device in some embodiments of the present application.

### DETAILED DESCRIPTION

Specific embodiments of the present application are described below. It should be noted that in the specific description of these embodiments, for a concise description, the present application may not describe in detail all of the features of the actual embodiments. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and extended, for a person of ordinary skill in the art related to the disclosure of the present application, some design, manufacture or production changes made on the basis of the technical disclosure of the present disclosure are only conventional technical means, and should not be construed that the content of the present disclosure is insufficient.

Unless otherwise defined, the technical or scientific terms used in the claims and the description should be as they are usually understood by those possessing ordinary skill in the technical field to which they belong. The terms “first”, “second” and similar words used in the present application and the claims do not denote any order, quantity, or importance, but are merely intended to distinguish between different constituents. The terms “one” or “a/an” and similar terms do not express a limitation of quantity, but rather that at least one is present. The terms “include”, “comprise”, or similar terms indicate that an element or object preceding the term “include” or “comprise” encompasses elements or objects and equivalent elements thereof listed after the term “include” or “comprise”, and does not exclude other elements or objects. The term “connect”, “connected” or a similar word is not limited to physical or mechanical connection, and is not limited to direct or indirect connection.

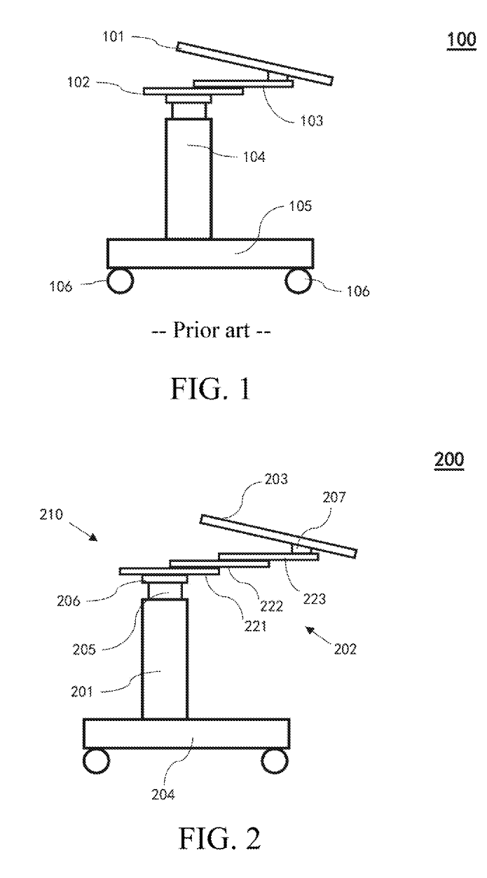

An adjustable operation panel is included in the prior art, for example an operation panel with functionality of telescoping and/or rotation in a horizontal direction, which can better meet the needs of users. FIG. 1 shows a schematic diagram of an apparatus 100 including an operation panel 101 in the prior art. As shown in FIG. 1, the apparatus 100 includes an operation panel 101, a first support arm 102, a second support arm 103, and a column 104.

The first support arm 102 may be connected to the column 104, so that the column 104 serves a support function for the entire apparatus. The second support arm 103 is connected to the operation panel 101. The first and second support arms may be telescopically connected by means of a mechanism such as a guide rail. Thus, when the second support arm 103 is operated to perform a telescopic motion with respect to the first support arm 102, the operation panel 101 provided on the second support arm 103 can also telescope, so that the position of the operation panel 101 with respect to a user can be adjusted without moving the column 104.

It can be understood that FIG. 1 shows a brief schematic view of the apparatus 100. The apparatus 100 in the prior art may include other components. For example, a component such as a display may also be provided on the operation panel 101. Alternatively, the operation panel 101 itself may serve as a platform for other devices to be fixed to, placed on, etc. Further, in addition to the column 104, the support structure may further include a base 105 and wheels 106 disposed below the base 105.

FIG. 1 shows an extended state of the first and second support arms. At this time, the weights of the operation panel 101 and the second support arm 103 act on the first support arm through a section (for example, part of guide rails) at which the first and second support arms are connected to each other. It can be understood that the shorter the length of the section, the greater the gravitational force experienced by the section per unit distance, and the more likely it is to cause damage to the connecting section. Therefore, in consideration of reliability and safety of the apparatus, it is necessary to ensure that the extension distance of the second support arm 103 can meet safety requirements. Of course, if limitations in size are not taken into consideration, the length of each of the first and second support arms may be set longer. However, setting the length too long may affect miniaturization of the apparatus, which is disadvantageous for transportation and use by users. Due to the above two factors, the stroke of the operation panel with a telescopic function is usually very limited.

In view of at least the above factors, improvements are proposed in some embodiments of the present application. Referring first to FIG. 2, an apparatus 200 including an operation panel assembly 210 in some embodiments of the present application is shown. As shown in FIG. 2, the operation panel assembly 210 may include a support assembly 201, an arm assembly 202, and an operation panel assembly 203.

The arm assembly 202 may include a first arm 221, a second arm 222, and a third arm 223. The first arm 221 is connected to the support assembly 201. The second arm 222 and the third arm 223 are telescopically connected with respect to the first arm 221. The operation panel 203 and the third arm 223 are connected.

Such an arrangement enables the operation panel 203 to be telescoped by movement of the three arms. On the one hand, the three arms can provide a larger stroke for the operation panel 203. On the other hand, the three arms can also ensure sufficient reliability of the device while maintaining a larger telescopic stroke. Specifically, referring to FIG. 2, the weights of the operation panel 203 and the third arm 223 act on the second arm 222 through a section (e.g., part of guide rails) at which the second and third arms are connected to each other. The weights of the operation panel 203 and the second and third arms act on the first arm 221 through a section (e.g., part of guide rails) at which the first and second arms are connected to each other. Since the three arms in the embodiments of the present application provide a larger telescopic stroke, even if the lengths of the two sections are appropriately extended (that is, the telescopic strokes of the second arm and the third arm are shortened), it can be ensured that the total telescopic stroke meets the requirements. Thus, the stability and reliability of the system are improved.

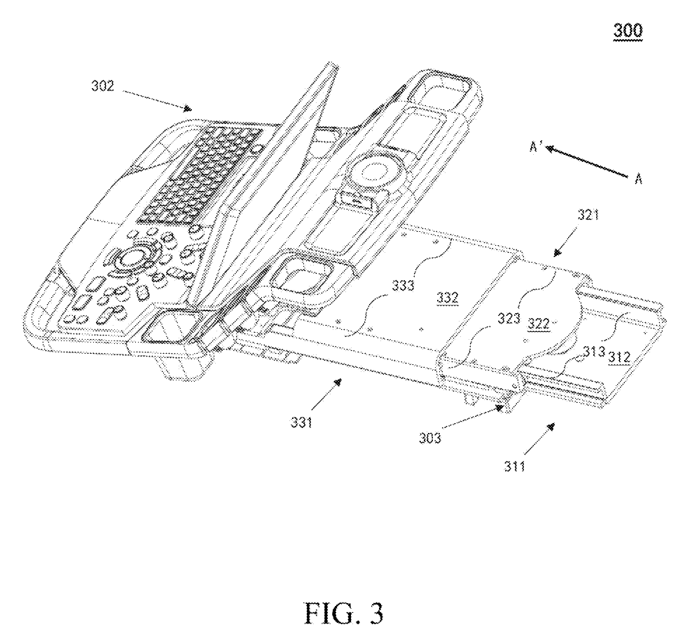

Preferred implementations of the operation panel assembly will be further described below. Referring to FIG. 3, a perspective view of an operation panel assembly 300 in further embodiments of the present application is shown.

The operation panel assembly 300 includes an arm assembly and an operation panel 302. The arm assembly may include a first arm 311, a second arm 321, and a third arm 331. As shown in FIG. 3, the first arm 311, the second arm 321, and the third arm 331 may be connected by means of guide rails. In some embodiments, the first arm 311 may include a first flat plate portion 312 and a first pair of oppositely disposed side walls 313. Similarly, the second arm 321 may include a second flat plate portion 322 and a second pair of oppositely disposed side walls 323. The third arm 331 may include a third flat plate portion 332 and a third pair of oppositely disposed side walls 333. The provision of the flat plate portions ensures that the three arms have higher mechanical strength and can be stacked on top of and support one another in a perpendicular direction. The first pair of side walls 313, the second pair of side walls 323, and the third pair of side walls 333 are disposed in a nested manner, which can guide telescoping of the second arm 321 and the third arm 331. Thus, the flat plate portions and the pairs of side walls ensure smooth and reliable telescopic motions of the second arm 321 and the third arm 331 in the horizontal direction.

It can be understood that the operation panel assembly 300 may likewise include other components, such as the support assembly 303 mentioned above. For the sake of simplicity, FIG. 3 does not show the entire support assembly 303 in detail, but shows only a part thereof that is connected to the first arm 311.

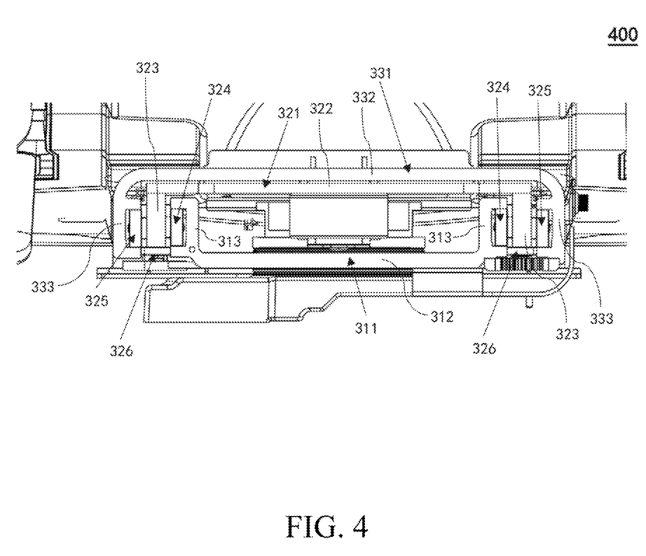

In order to more clearly describe the connection relationship among the first arm 311, the second arm 321, and the third arm 331, reference is further made to FIG. 4. FIG. 4 shows a partial schematic view of the connection among the three arms of the operation panel of FIG. 3 viewed in direction A-A’. In the embodiment shown in FIG. 4, the first arm 311, the second arm 321, and the third arm 331 are sequentially disposed from the inside to the outside in a nested manner. The first pair of side walls 313 of the first arm 311 are located on both sides of the first flat plate portion 312 and extend upward. Thus, the first flat plate portion 312 can be connected to the support assembly 303 without being blocked by other portions. Further, the pairs of side arms of the second arm 321 and the third arm 331 extend downward to form downward notches, respectively. Thus, the second arm 321 and the third arm 331 can be sequentially engaged onto the first arm 311, which achieves stable nesting. It can be understood that the case where the first to third arms are nested from the inside to the outside is described in the above embodiment, but the present application is not limited thereto. For example, the first to third arms may be nested from the outside to the inside, which will not be described in detail.

In some embodiments of the present application, further improvements are made to the connection among the three arms, taking into account at least the smoothness of the user pushing and pulling the third arm during use. In some embodiments, a first pair of guide rails and a first pair of guide wheels are disposed between the first pair of side walls and the second pair of side walls, respectively, the first pair of guide wheels being received in the first pair of guide rails; and a second pair of guide rails and a second pair of guide wheels are disposed between the second pair of side arms and the third pair of side arms, respectively, the second pair of guide wheels being received in the second pair of guide rails.

Specifically, FIG. 4 shows an implementation of the above embodiment. The first pair of side walls 313 include a first pair of guide rails (i.e., portions of the first pair of side walls 313 protruding outward in the figure), a first pair of guide wheels 324 are provided on the side of the second pair of side walls 323 facing the first pair of guide rails, and the first pair of guide wheels 324 are received in the first pair of guide rails. The third pair of side walls 333 include a second pair of guide rails (i.e., portions of the third pair of side walls 333 protruding inward in the figure), a second pair of guide wheels 325 are provided on the side of the second pair of side walls 323 facing the second pair of guide rails, and the second pair of guide wheels 325 are received in the second pair of guide rails 334.

According to the above implementation, when the second arm 321 is moved relative to the first arm 311 and the third arm 331 is moved relative to the second arm 321, the frictional force can be reduced to the minimum extent to avoid jamming.

It can be understood that the above embodiment may also be implemented in other specific ways. For example, the first pair of guide rails may be disposed on the side of the second pair of side walls facing the first arm, and the first pair of guide wheels may be disposed at corresponding positions of the first arm. Similarly, the second pair of guide rails may be disposed on the side of the second pair of side walls facing the third arm, and the second pair of guide wheels may be disposed at corresponding positions of the third arm. This can likewise guide the movement of the second arm and the third arm.

It should be noted that in the view of FIG. 4, only one set of second guide wheels and one set of third guide wheels are shown. It can be understood that the second guide wheels and third guide wheels may be disposed as multiple sets along the length direction of the arms (i.e. in direction A-A’ of FIG. 3) to provide uniform support and guidance for the movement of the arms.

Continuing to refer to FIG. 4, in an optional embodiment, the second pair of side walls 323 further includes a third pair of guide wheels 326, and the third pair of guide wheels 326 are disposed at bottoms of the second pair of side walls 323 and between the first pair of side walls 313 and the third pair of side walls 333. According to such an implementation, it is possible to further ensure the smoothness during a telescoping process of the second and third arms and to reduce the frictional force during the movement. Moreover, it is also possible to avoid shakes in the left-right direction during the telescoping process of the arms. Likewise, FIG. 4 shows only the case where the third pair of guide wheels 326 are provided as one set, but the third pair of guide wheels 326 may include a plurality of sets in the length direction of the arms.

It should be noted that, for the sake of simplicity, in the above of the present application, a plurality of embodiments are shown in FIG. 4 at the same time, but the present application is not limited thereto. In other words, the above embodiments are in a parallel relationship and can be freely combined under the teachings of the present disclosure, unless otherwise specified in the present application or the solutions obviously exclude such a combination.

So far, in the description of the above embodiments of the present application, a larger telescopic stroke is provided for the entire arm assembly by the movable connection between the second and third arms while the first arm is fixed, and the reliability of the movement can be ensured. Optionally, in the present application, the movable connection between the second and third arms is also optimized. In a further embodiment, the arm assembly further includes a synchronization apparatus through which the first arm, the second arm, and the third arm join one another to achieve synchronization of a telescoping process of the second arm and the third arm with respect to the first arm.

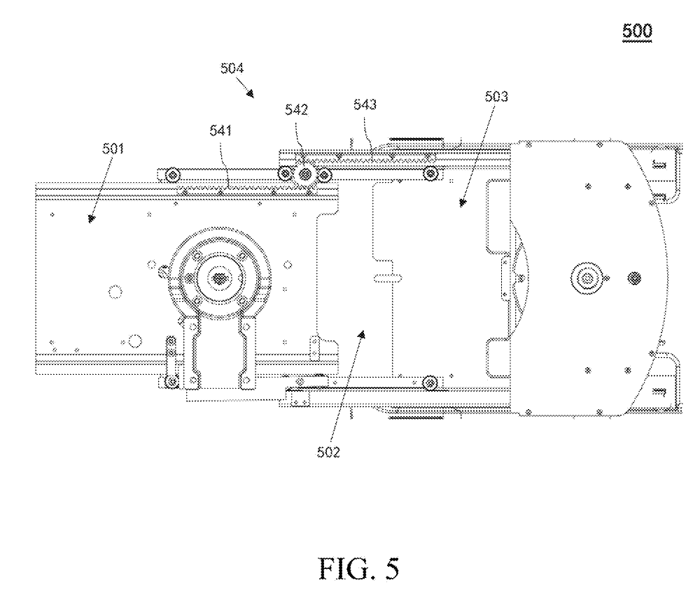

A detailed description will be provided below with reference to the drawings. Referring to FIG. 5, a bottom view of an arm assembly 500 in some embodiments of the present application is shown. For a first arm 501, a second arm 502, and a third arm 503 included in the arm assembly 500, reference may be made to the description of any one of the above embodiments of the present application, and the details thereof will not be further described here. In this embodiment, the arm assembly 500 further includes a synchronization apparatus 504. The synchronization apparatus 504 includes a first rack 541, a second rack 543, and a gear 542. The first rack 541 is disposed on the first arm 501. The second rack 543 is disposed on the third arm 503. As shown in the figure, the first rack 541 and the second rack 543 have a gap therebetween. The gear 504 is disposed on the second arm 502. Moreover, the gear 504 is engaged in the gap. Thus, the first arm 501, the second arm 502, and the third arm 503 join one another through the first rack 541, the gear 542, and the second rack 543. Thus, the telescoping process of the second arm 502 and the third arm 503 with respect to the first arm 501 (fixed arm) is synchronized.

By the above arrangement, when a user operates one of the movable arms, for example, the third arm b, in the arm assembly 500, the synchronization apparatus can drive the other movable arm, for example, the second arm 502, to move. In other words, the movable second arm 502 and the movable third arm 503 are thus synchronously moved or stationary, and there will be no case where the third arm 503 is pulled to move while the second arm 502 does not move. This can further improve the controllability of the operation panel assembly during the telescoping process. Since the two movable arms are moved or stopped at the same time, operating one of them means operating the other, thereby avoiding the occurrence of an abnormal sound caused by asynchronization or of an unreliable telescopic motion. Another advantage of the synchronization apparatus is that adjustment can be made to ensure that the second arm 502 and the third arm 503 synchronously reach the farthest distance when being operated to extend. The two arms can synchronously reach the required shortest distance when being operated to shorten.

It should be noted that synchronization means performing at the same time, and does not mean that the distances of movement (or speeds of movement) of the two arms are the same. A brief analysis is made with reference to FIG. 5. When the third arm is operated to extend, the third rack on the third arm can drive the gear on the second arm to roll. Since the gear on the second arm in turn joins the first rack on the first arm, the rolling of the gear on the second arm drives the movement of the second arm with respect to the first arm that is fixed differently. Considering movement at a certain moment, a gear may be regarded as a lever. Since the first arm is fixed, the fixed point (or fulcrum) of the lever is located at the point where the outer periphery of the gear joins the first rack (at a certain time, it can be considered that its speed is 0), and the point on the lever for urging the second arm to move is located at the connection point between the gear and the second arm, i.e. at the center of the gear. Thus, the length of the moment arm that drives the movement of the second arm can be understood as the radius of the gear. Similarly, the connection point between the lever and the third arm is the point at which the outer periphery of the gear joins the third rack, which may be regarded as the diameter of the gear. Generally, the moment arm of the lever for the third arm is twice the moment arm of the lever for the second arm. Accordingly, in this embodiment, the movements of the second and third arms are synchronized, and the relationship between the speed of movement of the third arm and the speed of movement of the second arm is also determined, the former being about twice the latter. The above analysis is intended to explain the synchronized movement of the present application more clearly, but not to limit the specific structure of the synchronization apparatus.

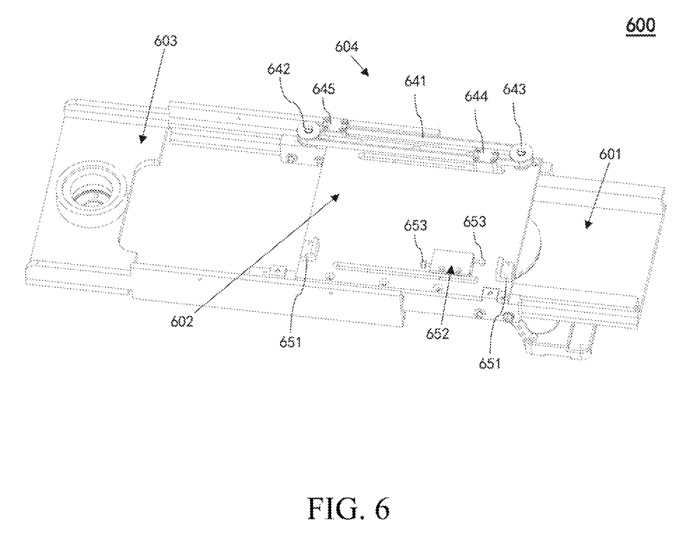

In further embodiments, the synchronization apparatus may further include other types of alternatives. Referring to FIG. 6, a bottom view of an arm assembly 600 in some other embodiments of the present application is shown. Similar to the foregoing, for a first arm 601, a second arm 602, and a third arm 603 included in the arm assembly 600, reference may be made to the description of any one of the above embodiments of the present application. In order to more clearly show the internal structure, the flat plate portion of the third arm 603 is removed from FIG. 6. The main difference is that, in this embodiment, the arm assembly 600 further includes a synchronization apparatus 604. As shown in FIG. 6, the synchronization apparatus 604 may include a transmission belt 641, a first transmission pulley 642, a second transmission pulley 643, a first fixing apparatus 644, and a second fixing apparatus 645. The first transmission pulley 642 and the second transmission pulley 643 are disposed on the second arm 602. The transmission belt 641 is tensioned by the first transmission pulley 642 and the second transmission pulley 643. The first fixing apparatus 644 is separately fixed to the first arm 601 and one side of the transmission belt 641. The second fixing apparatus 645 is separately fixed to the third arm 603 and the other side of the belt 641.

It should be noted that “one side” and “the other side” above may be understood to mean two parts into which the transmission belt 641 in a loop shape is divided by a line connecting the first and second transmission pulleys, where said one side is close to the first arm 601 and said other side is close to the third arm 603. Further, since the first arm 601 is fixedly disposed, it can thus be ensured that when the third arm 603 is extended or retracted, and after the first fixing apparatus 644 pulls the transmission belt 641, the first and second transmission pulleys connected to the transmission belt 641 are driven to move in the same direction, and finally the second arm 602 is driven to extend or retract synchronously.

In the embodiment of FIG. 6, for the synchronization effect of the second and third arms achieved by the synchronization apparatus and the proportional relationship of the speeds of movement of the second and third arms, reference may be made to the analysis with respect to FIG. 5. To be specific, said one side of the transmission belt and said other side of the transmission belt may be similar to the first and second racks of FIG. 5, and the first and second transmission pulleys may be analogous to the gear in the embodiment of FIG. 5. The details thereof will not be further described herein.

Further, any clamping apparatus such as a clamp in the prior art may be employed as the first and second fixing apparatuses, as long as it can ensure a fixed connection between the transmission belt and the arm and avoid occurrence of slipping. With respect to the transmission belt, in a preferred example, its inner surface may include a groove or a toothed structure. The outer surfaces of the first and second transmission pulleys may like include the structure described above so that slipping between the transmission belt and the transmission pulleys is less likely to occur during movement.

In some embodiments, the operation panel assembly may further include a buffer assembly. The buffer assembly includes a first buffer member and a second buffer member that are oppositely disposed, and the first buffer member and the second buffer member are disposed on any one arm and any other arm among the first arm, the second arm, and the third arm, respectively. The provision of the buffer assembly can ensure that the arm assembly, when extended or shortened to its limit position, does not collide violently and cause damage to the apparatus.

In an optional embodiment, the first buffer member includes two first buffer members disposed at front and rear ends of the one arm, and the second buffer member is disposed on the other arm and located between the two first buffer members. In other words, the second buffer member provided on the other arm is disposed between the first buffer members of the one arm. Thus, in addition to the buffer function, the buffer assembly can provide the function of stroke limiting. That is, the stroke of the buffer assembly is limited to the distance between one of the two first buffer members and the other. Accordingly, the relative movement between the one arm and the other arm will also be limited by the buffer assembly.

It can be understood that the one arm described above may be any one of the first to third arms, and the other arm described above may be any another of the first to third arms. One of the implementations will be described with reference to FIG. 6.

Referring to FIG. 6, the buffer assembly may include two first buffer members 651 and may further include a second buffer member 652. In the example of FIG. 6, the one arm described above is selected as the second arm 602, and the other arm described above is selected as the first arm 601. Accordingly, two first buffer members 651 are disposed at two end portions of the second arm 602. The second buffer member 652 is disposed on the first arm 601 and located between the two first buffer members 651. Thus, the movement of the second arm 602 will drive the two first buffer members 651 to move with respect to the second buffer member 652. When the second arm 602 extends to a limit position, one of the two first buffer members 651 abuts against the second buffer member 652, which thereby prevents further extension while provides the function of impact buffering. Similarly, when the second arm 602 is shortened to the limit position, the other of the two first buffer members 651 abuts against the second buffer member 652, which thereby prevents further shortening and can provide the function of impact buffering. Thus, the buffer assembly achieves both the stroke limitation of the movement of the arm assembly and impact buffering.

It can be understood that FIG. 6 specifically shows the case where the first and second buffer members are provided on the second arm 602 and the first arm 601, respectively. However, as described above in the present disclosure, the above arrangement is not the only arrangement. In light of the teachings of the specific embodiment of FIG. 6, an arbitrary selection may also be made among the three arms. For example, the first buffer member may be disposed on the first arm, the second buffer member may be disposed on the second arm, or any other case may be adopted. In addition, there may be diverse arrangement of the buffer members. The second buffer member 652 shown in FIG. 6 includes two elastic strikers 653 for contacting the two first buffer members 651 to provide the function of buffering, but other manners may also be employed. For example, an elastic material, a spring, or the like may be selected for buffering, or buffering and limiting, of which the details will not be further described.

In further embodiments, the limiting effect of the buffer assembly can be further enhanced when the synchronization apparatus and the buffer assembly in any one of the embodiments of the present application are both selected to be disposed. Specifically, FIG. 6 will still be used as an example for exemplary description. The synchronization apparatus 604 can ensure that the movements of the second arm 602 and the third arm 603 are synchronized. In other words, when the position of movement of any one of the second arm 602 and the third arm 603 reaches a certain limit of the stroke, the other will also reach a limit. On this basis, in the arm assembly including the synchronization apparatus 604, it is possible to buffer and limit all of the three arms by buffering or limiting only any two of the three arms. In FIG. 6, buffering and limiting occur between the first and second arms, but it can be understood that after the second arm 602 reaches a limit position and is limited by the buffering assembly, the third arm can also be stably limited at this time. Thus, the mutual cooperation between the synchronization apparatus 604 and the buffer assembly is achieved.

Various implementations and effects of the operation panel assembly having a telescopic function have been described in various embodiments above. In further embodiments, the operation panel assembly of the present application may further have other components and related functions. An exemplary description is provided below.

Referring back to FIG. 2, in some embodiments, the support assembly of the operation panel assembly 210 may further include a base 204 and a lifting apparatus 205. The lifting apparatus 205 is connected to the first arm 221 to drive the operation panel 203 to lift by means of the first arm 221, the second arm 222, and the third arm 223. Thus, when using the operation panel 203, the user can adjust not only the front-to-back distance of the operation panel 203 but also the height thereof.

For the specific configuration of the lifting apparatus 205, reference may be made to any method known in the art. For example, it may be achieved by means of a gas spring. Alternatively, it may be realized by means of a motor drive. It can be understood that the lifting apparatus 205 may further include a device such as a switch or the like to facilitate the user's adjustment for lifting, of which the details will not be further described here. The lifting apparatus 205 is connected to the base 204 and the first arm 221, and in addition to lifting, provides the function of supporting the operation panel 203.

Further, in some examples, a part of the structure of the operation panel assembly 201 of the present application is rotatable. For example, the operation panel 203 and the third arm 223 may be rotatably connected. In further examples, the support assembly 201 and the first arm 221 may be rotatably connected. In some other embodiments, while the operation panel 203 and the third arm 223 are rotatably connected, the support assembly 201 and the first arm 221 are further rotatably connected.

The rotatable operation panel 203 can further meet the user's needs for different angles of the operation panel. In particular, in an implementation in which both the first arm 221 and the operation panel 203 are configured to be rotatable, rotation at a larger angle can be achieved to meet the user's needs in more usage environments. To be specific, it is generally not appropriate for the rotatable operation panel provided in the prior art to have a very large rotation angle. Part of the reason is that a large rotation angle affects the reliability of the device, may reduce the stability of the connection between the operation panel and the arm, may also affect the lifetime of cables inside the panel, and so on. In the above embodiment of the present application, the rotation stroke is separately allocated to the first arm 221 and the operation panel 203, and thus the rotation angle is compensated by the first arm 221 without affecting the reliability of the operation panel 203 and the third arm 223 and the stability of the electrical connection inside the operation panel.

The rotatable connection may be achieved by means of any rotation mechanism. For example, as shown in FIG. 2, the support assembly 201 and the first arm 221 are rotatably connected by means of a first rotation mechanism 206. The operation panel 203 and the third arm 223 are rotatably connected by means of a second rotation mechanism 207. The first rotation mechanism 206 may include at least two portions that are disposed on the support assembly 201 and the first arm, respectively. Similarly, the second rotation mechanism 207 may likewise include at least two portions that are disposed on the operation panel 203 and the third arm 223, respectively. Thus, the rotatable connection is achieved.

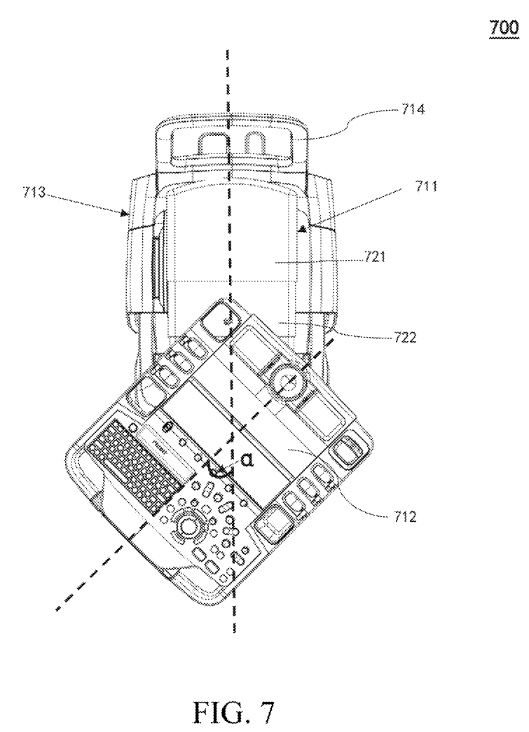

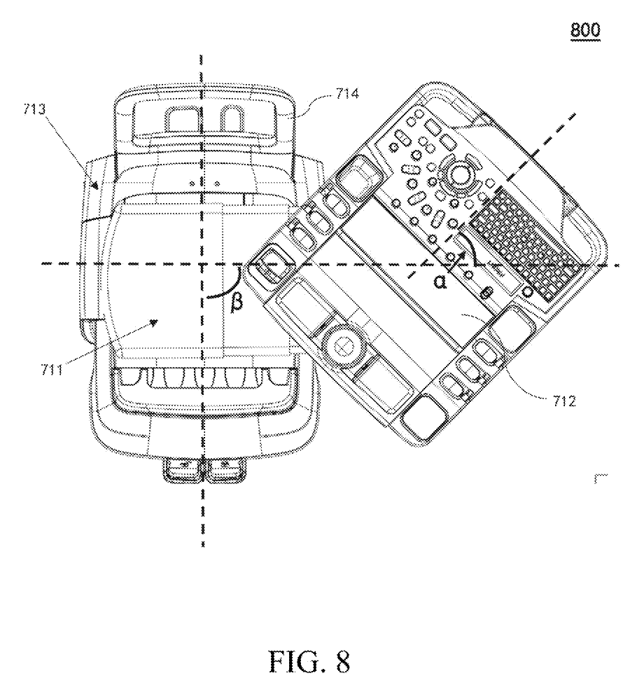

Referring to FIGS. 7 and 8, a rotatable state of the operation panel assembly in some embodiments of the present application will be described in further detail below. FIG. 7 shows a schematic view 700 of an operation panel assembly in a first state in some embodiments of the present application. FIG. 8 shows a schematic view 800 of the operation panel assembly in a second state in some embodiments of the present application.

Referring first to FIG. 7, the operation panel assembly includes an arm assembly 711, an operation panel 712, and a support assembly 713. For the specific structures of the above components, reference may be made to the description of any one of the above embodiments of the present application, and the details thereof will not be further described here. Optionally, the operation panel assembly may further include an attachment apparatus 714. As shown in the figure, the attachment apparatus 714 is connected to the support assembly 713 and extends upward to a height no lower than the height of the operation panel 712. The attachment apparatus 714 is shown in FIG. 7 in the form of a rear handle. Thus, when the user needs to move the operation panel assembly and the entire apparatus including the assembly, the user can operate the rear handle to do so.

FIG. 7 shows a rotation state of the operation panel 712 with respect to the arm assembly 711. In the case where the arm assembly 711 is not configured to be rotatable relative to the support assembly 713, the operation panel 712 is configured to be rotatable at ±α°. For human-machine interaction using the operation panel 712, if the body is not directly in front of the operation panel assembly, at this time, it is not necessary to laboriously move the entire operation panel assembly, but to adjust only the angle of the operation panel 712. However, as described above, the angular range cannot generally be configured to be excessively large, so as to avoid a decrease in connection reliability of the operation panel 712 or damage to internal electrical connections, or the like. As a result, in some usage scenarios, the angle of the operation panel 712 is still not optimal for user operation even if the operation panel is rotated.

FIG. 8 shows a rotation state of the operation panel 712 with respect to the arm assembly 711, and a rotation state of the arm assembly 711 with respect to the support assembly 713. The rotation state of the operation panel 712 with respect to the arm assembly 711 may be within a certain range, for example, at ±α°, as described with reference to FIG. 7. The main difference of the embodiment of FIG. 8 is that the arm assembly 711 is likewise configured to be rotatable. For example, in FIG. 8, the arm assembly 711 is rotated by β° with respect to the support assembly 713. Since the operation panel 712 is connected to the arm assembly 711, it means that the rotational stroke of the operation panel 712 with respect to the support assembly 713 is increased by ±β° from ±α° to ±(α+β)°. This will be able to meet the user's needs in more scenarios. Furthermore, since a part of the above stroke is brought about by the rotation of the arm assembly, the reliability of the operation panel 712 can be largely maintained.

Further, the inventors have realized that in an embodiment where an arm assembly having three arms is included, a farther extension stroke provided by the arm assembly can better cooperate with an implementation in which the operation panel and the arm assembly can both rotate. Referring to FIGS. 7 and 8, the arm assembly 711 may include a first arm 721, a second arm 722, and a third arm (located below the operation panel 712 and not shown). In some embodiments, the extension strokes of the second arm 722 and the third arm are configured such that rotational steric hindrance of the attachment apparatus 714 to the operation panel can be eliminated. To be specific, in the above embodiment, the extension strokes brought by the second arm 722 and the third arm can ensure that the operation panel 712 is sufficiently far away from the attachment apparatus 714. In this way, even at some relative limit positions, for example, when the arm assembly is rotated to an angle closest to the attachment apparatus while the operation panel is also rotated to an angle closest to the attachment apparatus, the operation panel is still not blocked by the attachment apparatus. Thus, the applicability of the operation panel with a large rotation angle is ensured.

On the basis of the rotatable connection between the operation panel and the third arm, some examples of the present application further provide a locking method for the rotational connection. To be specific, in an example in which the operation panel and the third arm are rotatably connected, the operation panel assembly may further include: a first locking member disposed between the operation panel and the third arm, the first locking member being operable to lock the operation panel and the third arm, so as to achieve rotational locking between the operation panel and the third arm. Such an arrangement can better meet the user's needs. For example, after the user rotates the operation panel to an appropriate angle, the operation panel can be locked with respect to the third arm by operating the first locking member, thereby positioning the angle of the operation panel and avoiding unexpected rotational movement during usage.

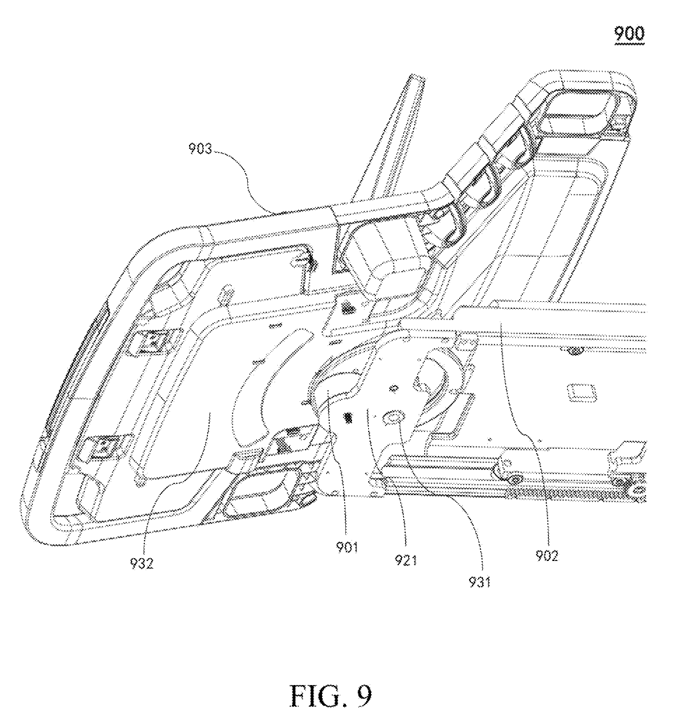

It can be understood that for the specific configuration of the first locking member itself, reference may be made to any locking mechanism known in the art, such as an electromagnetic or mechanical locking mechanism. The first locking member may further include a plurality of discrete components, such as some components distributed on the operation panel and some components on the third arm, and may further include other components for controlling the first locking member, such as a switch, a cable and the like, which will not be further enumerated. An exemplary description will be provided below with reference to FIG. 9.

Referring to FIG. 9, a partial schematic view of an operation panel assembly 900 including a first locking member in some embodiments of the present application is shown. It can be understood that, for components of the operation panel assembly 900 other than specific components to be described in detail below, reference may be made to the description of any one of the above embodiments herein.

As shown in FIG. 9, the operation panel assembly 900 includes a first locking member 901, a third arm 902, and an operation panel 903. In the embodiments shown in FIG. 9, the first locking element 901 is disposed between the third arm 902 and the operation panel 903. In an optional solution, the first locking member 901 is an electromagnetic locking apparatus fixedly mounted on a base plate 921 below the third arm 902. The operation panel 903 is rotatably connected to the third arm 902 by means of a rotating shaft 931. Moreover, the bottom surface of the operation panel 903 includes a ferromagnetic top plate 932. The ferromagnetic top plate 932 and the electromagnetic first locking member 901 are disposed oppositely. Thus, when energized, the first locking member 901 can generate a sufficiently strong magnetic field to attract and engage with the top plate 932. At this time, the bottom plate 921, the first locking member 901, and the top plate 932 are locked with one another. Accordingly, locking between the third arm 902 and the operation panel 903 is thereby achieved, which prevents rotation of the operation panel 903 with respect to the third arm 902.

The above is exemplary description of the first locking member 901 and its cooperating components. However, the present application is not limited thereto, and any apparatus that can be controlled by a switch for locking between the third arm 902 and the operation panel 903 is allowed, for example, a mechanical locking mechanism.

Furthermore, on the basis of the inclusion of a telescopic arm assembly, some examples of the present application further provide a locking method for the telescoping arm. To be specific, in some embodiments, the operation panel assembly of the present application further includes a second locking member disposed between the support assembly and the second arm, and the second locking member is operable to lock the support assembly and the second arm, so as to achieve telescopic locking of the second arm.

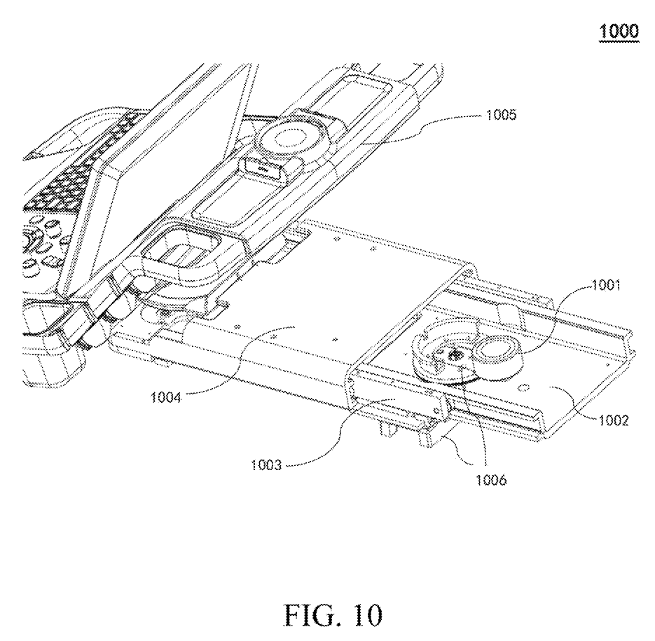

Further, in an embodiment where the support assembly and the first arm are configured to be rotatably connected, the second locking member can achieve rotational locking between the first arm and the support assembly while telescopically locking the second arm. The reason is that the second arm and the first arm are fixedly connected in a rotational direction. Accordingly, locking between the second arm and the support assembly means locking between the first arm and the support assembly in terms of the rotational function. Thus, in the present application, by providing the second locking member between the second arm and the support assembly, both telescopic locking of the second arm as well as rotational locking of the arm with respect to the support assembly can be achieved by only one set of locking members without providing another set of locking members. In this way, it is possible to reduce the cost and improve the reliability of the device, and to simplify the workflow of the user operation. A schematic and detailed description will be provided below with reference to FIG. 10. FIG. 10 shows a partial schematic view of an operation panel assembly 1000 including a second locking member in some embodiments of the present application. It can be understood that, for components of the operation panel assembly 1000 other than specific components to be described in detail below, reference may be made to the description of any one of the above embodiments herein.

As shown in FIG. 10, the operation panel assembly 1000 includes a second locking member 1001, a first arm 1002, a second arm 1003, a third arm 1004, an operation panel 1005, and a support assembly 1006. It should be noted that, in order to enable clearer description of the internal structure of the operation panel assembly 1000, a part of the flat plate portion of the second arm 1003 is removed from the view of FIG. 10. In the embodiments shown in FIG. 10, the second locking member 1001 is disposed between the second arm 1003 and the support assembly 1006. When the second locking member 1001 is energized, it can lock telescoping between the support assembly 1006 and the second arm 1003. In an optional solution, the second locking member 1001 is an electromagnetic locking apparatus fixedly mounted to the top end of the support assembly 1006 and disposed opposite to the flat plate portion of the second arm 1003.

In some embodiments, the top end of the support assembly 1006 is configured to include the structure of a rotating shaft, and the support assembly is rotatably connected to the first arm 1002. Thus, the arm assembly constituted by the first, second, and third arms, as a whole, is rotatably connected to the support assembly 1006. Accordingly, when the second locking member 1001 achieves the locking between the support assembly 1006 and the second arm 1003, not only telescopic locking but also rotational locking can be achieved. Thus, both telescopic locking of the second arm 1003 with respect to the support assembly 1006 and rotational locking of the arm assembly with respect to the support assembly 1006 can be achieved with only one second locking member 1001.

The inventors have further recognized that a combination of solutions in the above embodiments of the present application will be able to provide a greater contribution to enhancement of the performance of the operation panel assembly and improvement of the user experience than the simple combination itself. Exemplary examples will be provided below.

In some solutions, the operation panel assembly (e.g., the operation panel assembly 1000 of FIG. 10) includes both the synchronization apparatus described in the above embodiments and the second locking member. Accordingly, when telescopic locking between the second arm and the support assembly is achieved by using the second locking member, telescopic locking of the third arm may be achieved due to the presence of the synchronization apparatus. The reason is that the synchronization apparatus can ensure that the telescoping of the second and third arms is synchronized, and the telescoping movement of one of the arms drives the other one, and locking one of the arms can also cause the other arm to be locked. Thus, there is no need to provide two telescopic locking apparatuses for respectively locking two arms.

In some solutions, on the premise that the operation panel assembly includes both the first locking member described in the above embodiments and the second locking member (e.g., an embodiment in which both the operation panel assemblies of FIGS. 9 and 10 are included), a switch apparatus may be configured to control both locking members. Thus, when a user operates the operation panel to perform rotational locking or unlocking, the user does not need to consider how to separately control locking/unlocking between the operation panel and the third arm, or locking or unlocking between the arm assembly and the support assembly, and needs only to control a certain switch to achieve locking or unlocking at both points, thus improving the working efficiency.

In some solutions, on the premise that the operation panel assembly includes both the first locking member described in the above embodiments and the second locking member (e.g., an embodiment in which both the operation panel assemblies of FIGS. 9 and 10 are included), and includes a synchronization apparatus, both rotation and telescoping can be controlled by controlling the first locking member and the second locking member through a switch apparatus. The experience in operation and working efficiency of the user can be further improved.

So far, various operation panel assemblies have been described in the above embodiments of the present application. The operation panel assemblies may be applied to different devices. In some embodiments, a medical device including an operation panel assembly described in any one of the above embodiments is disclosed in the embodiments of the present application.

Optionally, the medical device described above may include at least one of a medical cart and an ultrasound imaging device. The medical cart, as a carrying mechanism, may be used to carry various medical apparatuses such as monitors, portable ultrasound devices, electrocardiographs, etc. The medical cart equipped with the above operation panel assembly of the present application can be given more functions to improve the convenience in user operation. In further implementations, the operation panel assembly described in any one of the above embodiments may be used as part of an ultrasound imaging device. An exemplary description is provided below.

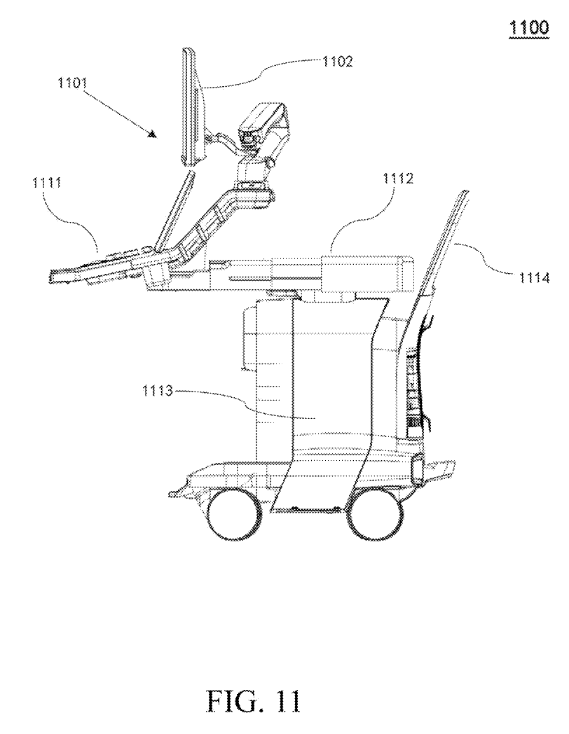

Referring to FIG. 11, a schematic view of an ultrasound imaging device 1100 in some embodiments of the present application is shown.

The ultrasound imaging device 1100 may include an operation panel assembly 1101 and a display 1102 on the operation panel assembly 1101. For the specific arrangement of the operation panel assembly 1101, reference may be made to the description of any one of the above embodiments herein. For example, an operation panel 1111, an arm assembly 1112, a support assembly 1113, and a rear handle 1114 may be included. The operation panel 1111 provides a user interface for human-machine interaction, such as a touch screen, a keyboard, etc. The present application provides the user with a flexible way to adjust the operation panel assembly 1101. Thus, the user may operate the operation panel 1111 at different distances and angles, and observe medical information such as an ultrasound image on the display 1102.

It can be understood that the above is merely an exemplary description of the ultrasound imaging device 1100. For other structures of the device, reference may be made to any of those in the prior art. For example, a probe or other accessories may be further included.

The purpose of providing the above specific embodiments is to allow the disclosure of the present application to be understood more thoroughly and comprehensively; however, the present application is not limited to said specific embodiments. A person skilled in the art should understand that various modifications, equivalent replacements, changes and the like can be further made to the present application and should be included in the scope of protection of the present application as long as these changes do not depart from the spirit of the present application.

1. An operation panel assembly, comprising:
a support assembly;
an arm assembly comprising a first arm, a second arm, and a third arm; the first arm being connected to the support assembly; the second arm and the third arm being telescopically connected with respect to the first arm; and
an operation panel connected to the third arm.

2. The operation panel assembly according to claim 1, wherein the arm assembly further comprises:
a synchronization apparatus through which the first arm, the second arm, and the third arm join one another to achieve synchronization of a telescoping process of the second arm and the third arm with respect to the first arm.

3. The operation panel assembly according to claim 2, wherein the synchronization apparatus comprises:
a first rack disposed on the first arm;
a second rack disposed on the third arm, the first rack and the second rack having a gap therebetween; and
a gear disposed on the second arm and engaged in the gap; the first arm, the second arm, and the third arm join one another through the first rack, the gear, and the second rack.

4. The operation panel assembly according to claim 2, wherein the synchronization apparatus comprises:
a transmission belt tensioned by a first transmission pulley and a second transmission pulley disposed on the second arm;
a first fixing apparatus separately fixed to the first arm and one side of the transmission belt; and
a second fixing apparatus separately fixed to the third arm and the other side of the transmission belt.

5. The operation panel assembly according to claim 1, wherein the support assembly comprises:
a base; and
a lifting apparatus connected to the first arm to drive the operation panel to lift by means of the first arm, the second arm, and the third arm.

6. The operation panel assembly according to claim 1, wherein:
the operation panel and the third arm are rotatably connected; and/or
the support assembly and the first arm are rotatably connected.

7. The operation panel assembly according to claim 6, further comprising:
an attachment apparatus connected to the support assembly and extending upward to a height not lower than the height of the operation panel; extension strokes of the second arm and the third arm are configured such that rotational steric hindrance of the attachment apparatus to the operation panel can be eliminated.

8. The operation panel assembly according to claim 7, wherein the attachment apparatus comprises a rear handle.

9. The operation panel assembly according to claim 1, wherein:
the first arm comprises a first flat plate portion and a first pair of oppositely disposed side walls; the second arm comprises a second flat plate portion and a second pair of oppositely disposed side walls; the third arm comprises a third flat plate portion and a third pair of oppositely disposed side walls; and the first pair of side walls, the second pair of side walls, and the third pair of side walls are disposed in a nested manner to guide telescoping of the second arm and the third arm.

10. The operation panel assembly according to claim 9, wherein:
a first pair of guide rails and a first pair of guide wheels are disposed between the first pair of side walls and the second pair of side walls, respectively, the first pair of guide wheels being received in the first pair of guide rails; and a second pair of guide rails and a second pair of guide wheels are disposed between the second pair of side arms and the third pair of side arms, respectively, the second pair of guide wheels being received in the second pair of guide rails.

11. The operation panel assembly according to claim 10, wherein:
the second pair of side walls further comprises a third pair of guide wheels disposed at bottoms of the second pair of side walls and between the first pair of side walls and the third pair of side walls.

12. The operation panel assembly according to claim 1, further comprising:
a buffer assembly comprising a first buffer member and a second buffer member that are oppositely disposed, the first buffer member and the second buffer member being disposed on any one arm and any other arm among the first arm, the second arm, and the third arm, respectively.

13. The operation panel assembly according to claim 12, wherein:
the first buffer member comprises two first buffer members disposed at front and rear ends of the one arm, and the second buffer member is disposed on the other arm and located between the two first buffer members.

14. The operation panel assembly according to claim 1, wherein the operation panel and the third arm are rotatably connected, and the operation panel assembly further comprises:
a first locking member disposed between the operation panel and the third arm, the first locking member being operable to lock the operation panel and the third arm, so as to achieve rotational locking between the operation panel and the third arm.

15. The operation panel assembly according to claim 1, further comprising:
a second locking member disposed between the support assembly and the second arm, the second locking member being operable to lock the support assembly and the second arm, so as to achieve telescopic locking of the second arm.

16. The operation panel assembly according to claim 15, wherein the support assembly and the first arm are rotatably connected, whereby the second locking member achieves rotational locking between the first arm and the support assembly while telescopically locking the second arm.

17. A medical device, comprising an operation panel assembly according to any one of claims 1-16.

18. The medical device according to claim 17, comprising at least one of a medical cart and an ultrasound imaging apparatus.
